(19) United States
(12) Patent Application Publication
(10) Pub. No.: US 2020/0340893 A1
(43) Pub. Date: Oct. 29, 2020

(54) Hydrogel Particle-Enabled Large Volume Fluid Processing Device and System

(71) Applicant: CERES NANOSCIENCES, Manassas, VA (US)

(72) Inventors: ROBERTO JUAN BARBERO, Manassas, VA (US); Benjamin LEPENE, Manassas, VA (US); Ketul SHAH, Manassas, VA (US); Patrick ANDERSEN, Manassas, VA (US); Bob EISELE, Carlsbad, CA (US); Michael WILLIAMS, Carlsbad, CA (US)

(21) Appl. No.: 16/725,057

(22) Filed: Dec. 23, 2019

Related U.S. Application Data
(60) Provisional application No. 62/783,994, filed on Dec. 21, 2018.

(51) Int. Cl. G01N 1/40; G01N 33/52

(52) U.S. Cl. CPC G01N 1/4077; G01N 2001/4088; G01N 33/526

(57) ABSTRACT

A device configured to capture analytes from a large volume of fluid (e.g. more than 2 mL of urine, broth, or water), concentrate those analytes, and elute them into a small volume (e.g. 100 uL) for subsequent analysis is described. Use of the apparatus for more than 100×-1000× concentration of analytes. Hydrogel particles are specially engineered and designed to capture and retain specific analytes from the fluid and then exclude other, unwanted analytes. The device has three pieces, including a fluid filtration cup, a waste collection chamber, and an elution collection chamber.

### CONTINUITY

This application is a non-provisional application of provisional patent application No. 62/783,994, filed on Dec. 21, 2018, and priority is claimed thereto.

### FIELD OF THE PRESENT INVENTION

The present invention relates to the field of fluid processing, and more specifically relates to a hydrogel particle-enabled large volume fluid processing device configured to capture and retain specific analytes contained within large volumes of fluid.

### BACKGROUND OF THE PRESENT INVENTION

Currently, it is possible to capture, concentrate, and preserve low abundance analytes as long as they are presented within a low volume sample. However, in order to accomplish this same feat with a large volume sample, such as those over 2 mL or more, the use of expensive and large centrifuges are required. This, in turn, may take over an hour in order to separate the requisite target particles from the fluid.

If there were a device and system by which a large volume sample of a fluid could be analyzed, and by which low abundance analytes could be captured, concentrated, and preserved from the sample without the use of an expensive, large centrifuge, the process could be expedited and facilitated.

Thus, there is a need for a new system and apparatus configured to facilitate and expedite the capture, concentration, and preservation of low abundance analytes from a large volume fluid. Such an apparatus and system preferably employs a cup in communication with a vacuum, which pulls the large volume fluid across a filter membrane. The fluid is equipped with affinity capture hydrogel particles which facilitate the filtration process by capturing the desired analytes of the large volume fluid without the use of a centrifuge.

### SUMMARY OF THE PRESENT INVENTION

The present invention is a hydrogel particle-enabled large volume fluid processing system and device configured to facilitate the detection and capture of analytes contained within fluid such as urine.

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

The present invention will be better understood with reference to the appended drawing sheets, wherein:

FIG. 1 depicts a view of the large volume fluid processing device of the present invention.

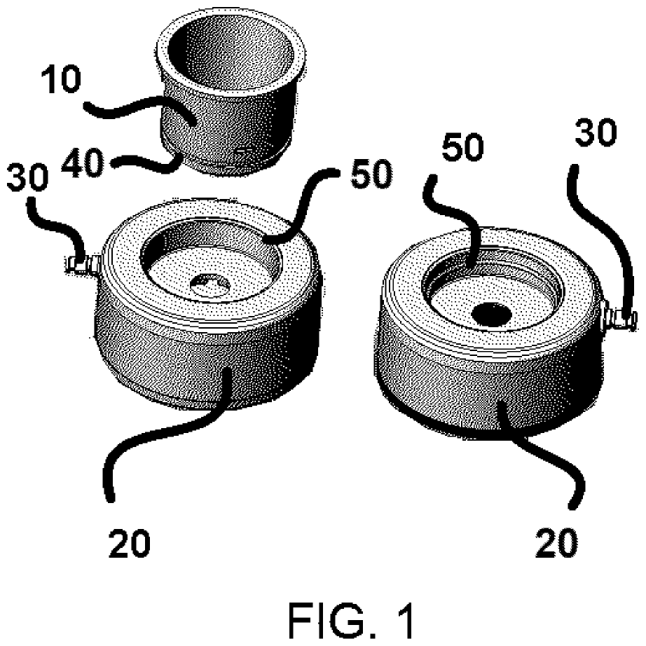

### DETAILED DESCRIPTION OF THE PREFERRED EMBODIMENT

The present specification discloses one or more embodiments that incorporate the features of the invention. The disclosed embodiment(s) merely exemplify the invention. The scope of the invention is not limited to the disclosed embodiment(s). The invention is defined by the claims appended hereto.

References in the specification to “one embodiment,” “an embodiment,” “an example embodiment,” etc., indicate that the embodiment described may include a particular feature, structure, or characteristic, but every embodiment may not necessarily include the particular feature, structure or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The present invention is a hydrogel particle-enabled large volume fluid processing device and system. The present invention is configured to facilitate the detection of various diseases and conditions. The device is equipped with a filtration cup (10), a base (20), a seal (50), and a vacuum port (30). The filtration cup (10) of the present invention is equipped with a membrane disposed at the bottom of the filtration cup (10) which is configured to facilitate the capture of analytes in contact with the hydrogel particles. The volume of fluid is disposed within the filtration cup (10), which is then placed atop the base (20). The filtration cup (10) establishes a firm seal to the base (20) via the seal (50) disposed within the base (20) or attached to the cup (10). In this manner, the filtration cup (10) is mated to the base (20) such that no leaks of suction are present. A vacuum hose is connected to the vacuum port (30). The base (20) channels the vacuum supplied by the vacuum hose to a hole disposed within the base (20), which efficiently pulls the large volume of fluid through the filtration cup (10), whereby the analytes are captured within the membrane. An array of pillars (40) are present within the filtration cup (10) which ensure that the membrane is maintained off of the bottom of the filtration cup (10). By ensuring that the filter remains off of the bottom of the filtration cup (10), the fluid flows evenly through the entire filter membrane and the prescribed projected area of the filter media remains functional.

In some embodiments of the present invention, the base (20) is equipped with a waste chamber which is preferably equipped with an absorbent material. The absorbent material is configured to capture fluid after it passes through the filtration cup (10) such that it may be easily disposed after use, and helps to reduce the possibility of spillage. In this particular embodiment the seal between the filtration cup (10) and the base (20) is created with an O-ring incorporated into the base cup (20). Other embodiments include one where an O-ring is incorporated into the filtration cup (10); one where an integral seal (i.e. overmolded) is built into the filtration cup (10); one where an integral seal (i.e. overmolded) is built into the base (20); one with a loose gasket or a gasket this is permanently attached to either the base (20) or the filtration cup (10) with adhesive (pressure sensitive or other type of adhesive); or one where the filtration cup (10) is integral with the filter housing.

### Applications of Use of the Present Invention:

Use of the present invention has been demonstrated in cases of the detection of Lyme Disease. For example, the present invention can be used to enrich the OspA antigen from urine in order to improve the limit of detection of an ELISA for OspA, and for a lateral flow assay specific to OspA. See experimental results below comparing the use of this device to a method that separates hydrogel particles from the urine with a centrifugation method.

Experimental design specifications of the apparatus and system of the present invention are preferably as follows:
Spike OspA into urine at four different concentrations: 50 pg/mL; 20 pg/mL; 5 pg/mL; 0 pg/mL.
   Using 40 mL urine volumes, captured and measured OspA from these samples using four different hydrogel particle methods.
      1. Separated hydrogel particles from urine using centrifugation and measured OspA using ELISA.
      2. Separated hydrogel particles from urine using prototype urine processing device and measured OspA using lateral flow assay.
      3. Separated hydrogel particles from urine using centrifugation and measured OspA using lateral flow assay.
      4. Separated Nanotrap particles from urine using prototype urine processing device and measured OspA using ELISA.
Ran each concentration on each method in duplicate.
Cutoff for detecting a sample=3×stdev (signal at 0 pg/mL)+average (signal at 0 pg/mL).
Both duplicates must have signal above cutoff in order for a sample to be above LOD.

### Experimental Design

Spiked OspA into urine at 4 different concentrations: 50 pg/mL; 20 pg/mL; 5 pg/mL; 0 pg/mL
   Using 40 mL urine volumes, captured and measured OspA using from these samples using four different Nanotrap particle methods
      1. Separated Nanotrap particles from urine using centrifugation and measure OspA using ELISA
      2. Separated Nanotrap particles from urine using prototype urine processing device and measured OspA using lateral flow assay
      3. Separated Nanotrap particles from urine using centrifugation and measured OspA using lateral flow assay
      4. Separated Nanotrap particles from urine using prototype urine processing device and measured OspA using ELISA
   Ran each concentration on each method in duplicate
   Cutoff for detecting a sample=3×stdev(signal at 0 pg/mL)+average(signal at 0 pg/mL)
   Both duplicates must have signal above cutoff in order for a sample to be above LOD

| | ELISA | LATERAL FLOW |
|---|---|---|
| CENTRIFUGATION | <5 pg/mL | <20 pg/mL |
| URINE PROCESSING | <5 pg/mL | <20 pg/mL |
| DEVICE | | |

It has been demonstrated that the device of the present invention can be used to enrich the TB LAM antigen from urine in order to improve the limit of detection of a Binax NOW TB LAM lateral flow assay.

Alternate embodiments of the present invention may include variations on the order of operations of use of the apparatus of the present invention. For example, in another iteration of the present invention, the particles could be attached to the membrane prior to addition of the fluid. Similarly, in another iteration of the present invention, the affinity capture moiety could be attached to the membrane prior to the addition of the fluid. Alternately, in another iteration of the present invention, the detection method can be a lateral flow assay that is inserted directly into stage 2 of the device and the elution buffer is delivered directly onto the device.

It should be understood that the fluid employed in use of the apparatus of the present invention can be any fluid that can be processed through a filter with the pore size used in the filtration cup. This fluid can be urine, water, buffer, broth, etc. Additionally, it should be noted that the suction strength of the vacuum connected to the vacuum port (30) of the present invention need not be any specific value, but is understood to be a negative pressure adequate to draw the employed fluid through the filtration cup (10) of the present invention during use.

Having illustrated the present invention, it should be understood that various adjustments and versions might be implemented without venturing away from the essence of the present invention. Further, it should be understood that the present invention is not solely limited to the invention as described in the embodiments above, but further comprises any and all embodiments within the scope of this application.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

I claim:

1. A device for capturing analytes from a volume of fluid larger than 2 mL, concentrating the analytes via a vacuum, and eluting the analytes for analysis comprising:
a base;
a filtration cup;
wherein said filtration cup contains a filter;
an array of pillars, said array of pillars present within said filtration cup;
wherein said array of pillars ensure said filter is maintained in a position up and away from a bottom of said filtration cup;
a seal;
wherein the volume of fluid is disposed within the filtration cup;
wherein said filtration cup is disposed atop said base;
wherein said seal is present between said filtration cup and said base, establishing a firm watertight seal between said filtration cup and said base; and
a vacuum port, said vacuum port disposed on said base.
